(19) United States
(12) Patent Application Publication
(10) Pub. No.: US 2023/0100459 A1
(43) Pub. Date: Mar. 30, 2023

(54) ELECTROSURGICAL INSTRUMENT WITH FIBER OPTIC ROTARY COUPLING

(71) Applicant: Cilag GmbH International, Zug (CH)

(72) Inventors: Carl J. Draginoff, Jr., Mason, OH (US); Chad P. Boudreaux, Cincinnati, OH (US); Thomas J. Watson, Cambridgeshire (GB); Robertus C. Meijer, Cambridge (GB)

(21) Appl. No.: 17/489,894

(22) Filed: Sep. 30, 2021

(51) Int. Cl. A61B 18/14; G02B 6/44

(52) U.S. Cl. CPC A61B 18/1445; G02B 6/4457; A61B 2017/2912

(57) ABSTRACT

A surgical instrument includes a body, a shaft assembly extending distally from and rotatably coupled to the body, an end effector extending distally from the shaft assembly, a fiber optic cable, and a rotary coupling assembly. The end effector may rotate with the shaft assembly relative to the body. The end effector includes a sensor assembly. The fiber optic cable extends proximally from the sensor assembly, along the shaft assembly, and into the body. The fiber optic cable includes a distal portion that rotates with the end effector, a proximal portion associated with the body, and a coiled portion interposed between the distal portion and the proximal portion. The rotary coupling assembly includes a handle-side body and a shaft-side body. The rotary coupling assembly can radially expand and contract the coiled portion of the fiber optic cable in response to relative rotation between the handle-side body and the shaft-side body.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 8,939,974, entitled “Surgical Instrument Comprising First and Second Drive Systems Actuatable by a Common Trigger Mechanism,” issued Jan. 27, 2015, the disclosure of which is incorporated by reference herein, in its entirety; U.S. Pat. No. 8,888,809, entitled “Surgical Instrument with Jaw Member,” issued Nov. 18, 2014, the disclosure of which is incorporated by reference herein, in its entirety; U.S. Pat. No. 9,161,803, entitled “Motor Driven Electrosurgical Device with Mechanical and Electrical Feedback,” issued Oct. 20, 2015, the disclosure of which is incorporated by reference herein, in its entirety; U.S. Pat. No. 9,877,720, entitled “Control Features for Articulating Surgical Device,” issued Jan. 30, 2018, the disclosure of which is incorporated by reference herein, in its entirety; U.S. Pat. No. 9,545,253, entitled “Surgical Instrument with Contained Dual Helix Actuator Assembly,” issued Jan. 17, 2017, the disclosure of which is incorporated by reference herein, in its entirety; and U.S. Pat. No. 9,526,565, entitled “Electrosurgical Devices,” issued Dec. 27, 2016, the disclosure of which is incorporated by reference herein, in its entirety.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4A depicts a side elevational view of a handle assembly of the electrosurgical instrument of FIG. 1, where the end effector is in an open and unfired state, where a portion of the handle assembly is omitted for purposes of clarity;

FIG. 4B depicts a side elevational view of the handle assembly of FIG. 4A, where the end effector is in a closed and unfired state, where a portion of the handle assembly is omitted for purposes of clarity;

FIG. 4C depicts a side elevational view of the handle assembly of FIG. 4A, where the end effector is in a closed and fired state, where a portion of the handle assembly is omitted for purposes of clarity;

FIG. 5A depicts a cross-sectional side view of the end effector of FIG. 2, where the end effector is in the open and unfired state, taken along line 5-5 of FIG. 2;

FIG. 5B depicts a cross-sectional side view of the end effector of FIG. 2, where the end effector is in the closed and unfired state, taken along line 5-5 of FIG. 2;

FIG. 5C depicts a cross-sectional side view of the end effector of FIG. 2, where the end effector is in the closed and fired state, taken along line 5-5 of FIG. 2;

FIG. 6 depicts a perspective view of another exemplary electrosurgical instrument;

FIG. 7 depicts a perspective view of a fiber optic rotary coupling assembly of the electrosurgical instrument of FIG. 6;

FIG. 8 depicts an exploded perspective view of the fiber optic rotary coupling assembly of FIG. 7;

FIG. 9 depicts an elevational side view of an inner spindle assembly of the fiber optic rotary coupling assembly of FIG. 7;

FIG. 10 depicts a perspective view of the inner spindle assembly of FIG. 9 coupled with a pair of fiber optic cables of the electrosurgical instrument of FIG. 6;

FIG. 11 depicts a perspective sectional view of a housing assembly of the fiber optic rotary coupling assembly of FIG. 7, taken along a centerline thereof;

FIG. 12A depicts a partial sectional view of the fiber optic rotary coupling assembly of FIG. 7 in a first rotational position such that a coiled portion of the pair of fiber optic cables of FIG. 10 are in a neutral position;

FIG. 12B depicts a partial sectional view of the fiber optic rotary coupling assembly of FIG. 7 in a second rotational position such that a coiled portion of the pair of fiber optic cables of FIG. 10 are in a radially expanded position;

FIG. 12C depicts a partial sectional view of the fiber optic rotary coupling assembly of FIG. 7 in a third rotational position such that a coiled portion of the pair of fiber optic cables of FIG. 10 are in a radially retracted position;

FIG. 13 depicts a perspective view of a second fiber optic rotary coupling assembly; and

FIG. 14 depicts a perspective view of the fiber optic rotary coupling assembly of FIG. 7 incorporated into an exemplary ultrasonic instrument.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

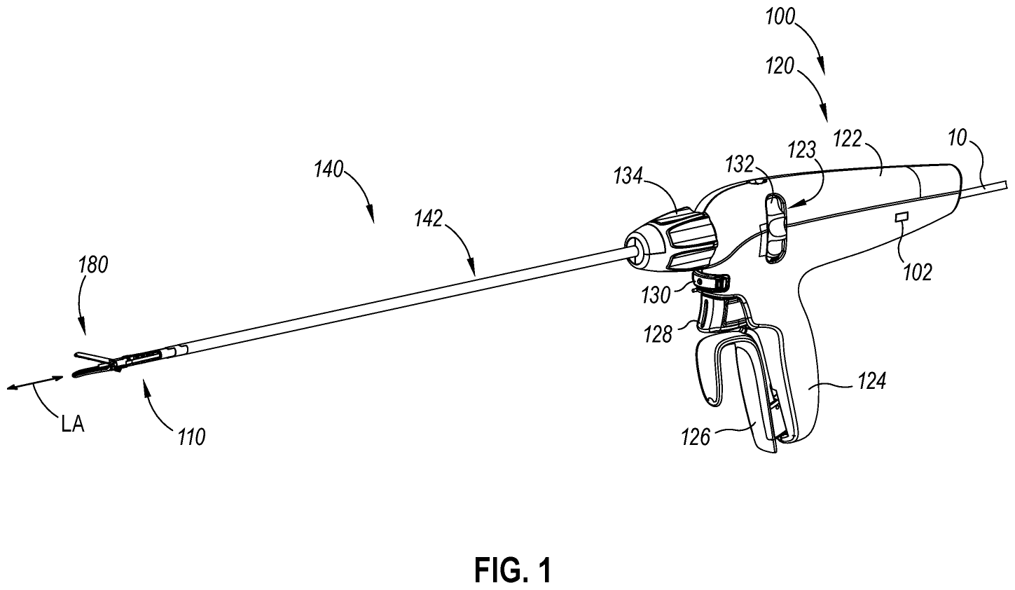

FIGS. 1-3C show an exemplary electrosurgical instrument (100). As best seen in FIG. 1, electrosurgical instrument (100) includes a handle assembly (120), a shaft assembly (140), an articulation assembly (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to seal or weld tissue by applying bipolar radio frequency (RF) energy to tissue. However, it should be understood electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a power source (not shown) via power cable (10).

The power source may be configured to provide all or some of the electrical power requirements for use of electrosurgical instrument (100). Any suitable power source may be used as would be apparent to one skilled in the art in view of the teachings herein. By way of example only, the power source may comprise a GEN04 or GEN11 sold by Ethicon Endo-Surgery, Inc. of Cincinnati, Ohio. In addition, or in the alternative, the power source may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to a power source via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrode surfaces (194, 196) of jaws (182, 184), respectively.

Body (122) of handle assembly (120) defines an opening (123) in which a portion of articulation control (132) protrudes from. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) is configured to bend articulation section (110) in order to drive deflection of end effector (180) from the longitudinal axis (LA) defined by shaft assembly (140). Articulation control (132) and articulation section (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

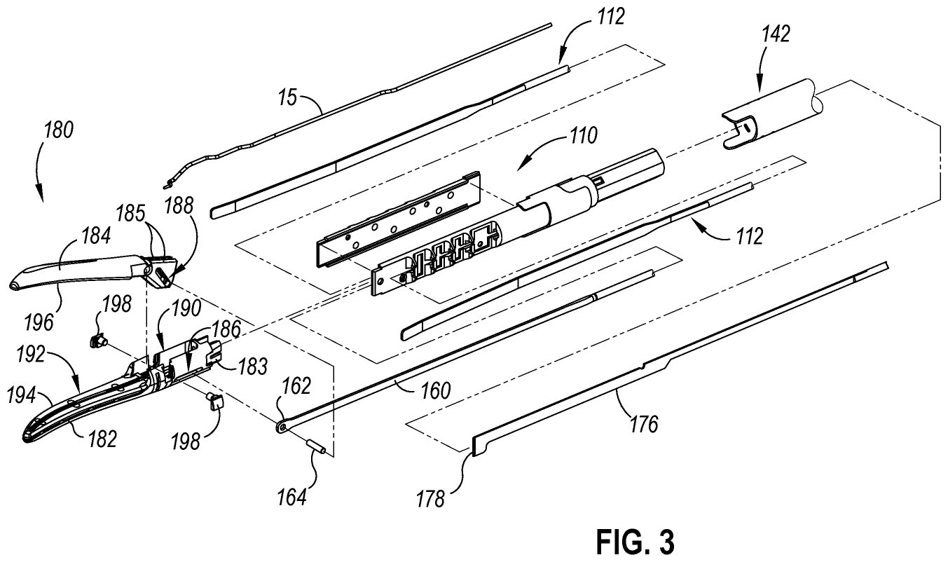

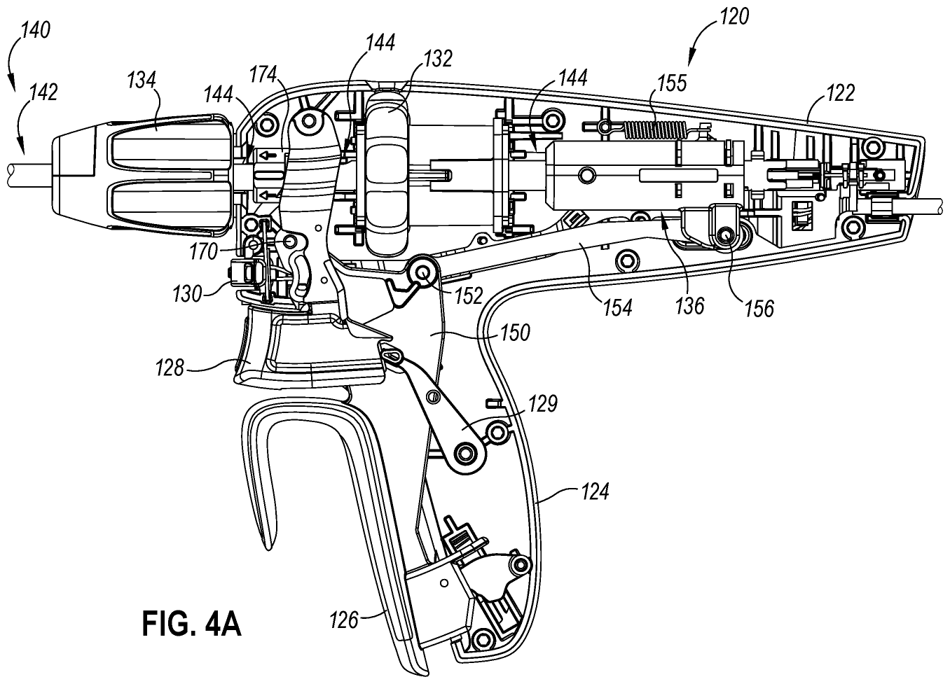

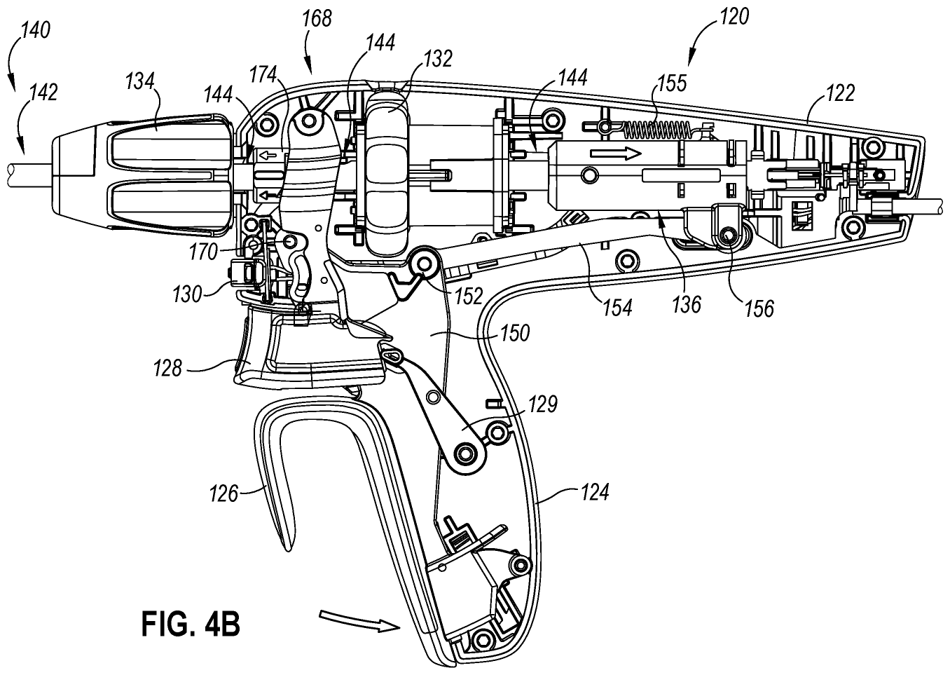

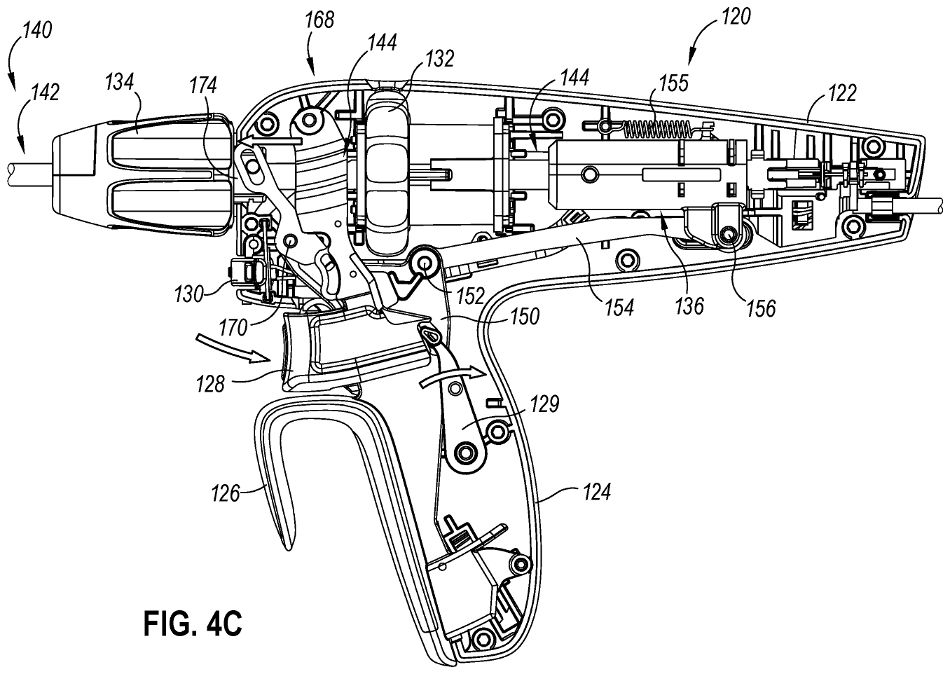

Shaft assembly (140) includes distal portion (142) extending distally from handle assembly (120), and a proximal portion (144) (see FIGS. 4A-4B) housed within the confines of body (122) of handle assembly (120). As best shown in FIG. 3, shaft assembly (140) houses a jaw closure connector (160) that couples jaw closure trigger (126) with end effector (180). Additionally, shaft assembly (140) houses a portion of knife member extending between distal cutting edge (178) and knife trigger (128). Shaft assembly (140) also houses actuating members (112) that couple articulation assembly (110) with articulation control (132); as well as an electrical connecter (15) that operatively couples electrode surfaces (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrode surface (194, 196).

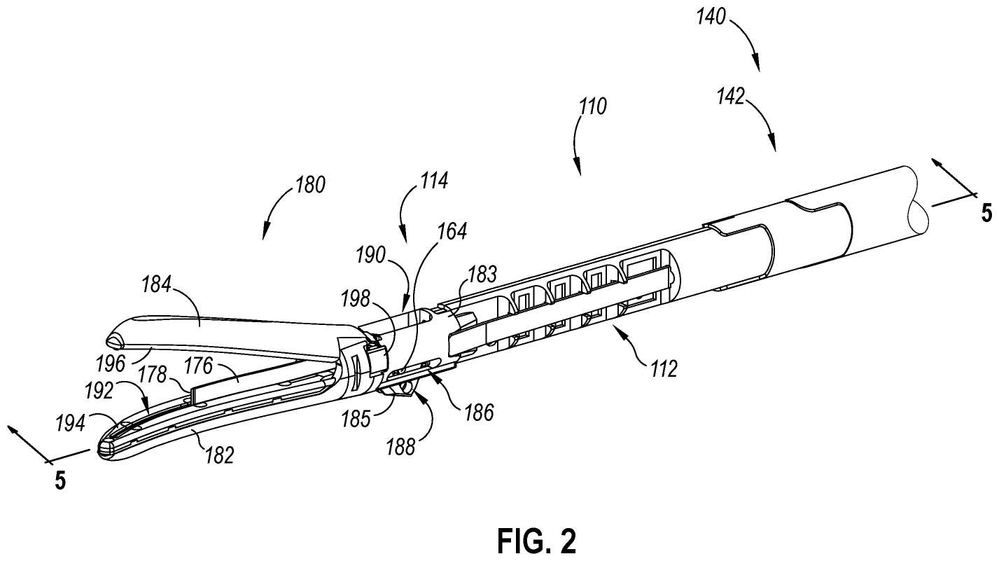

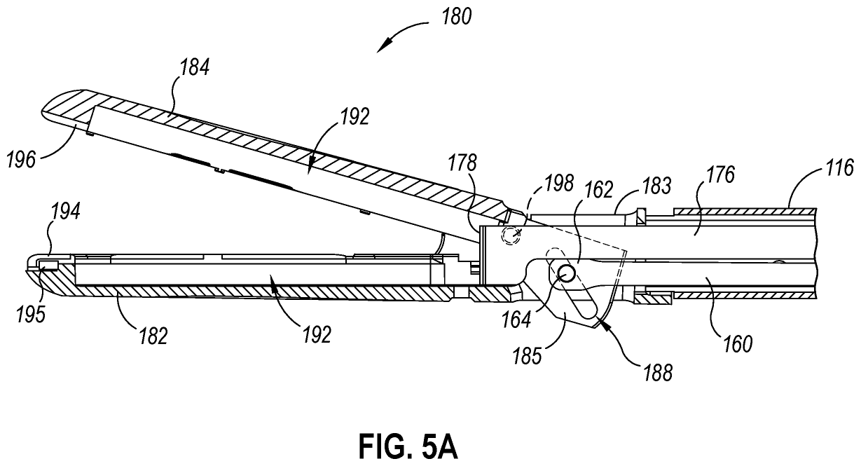

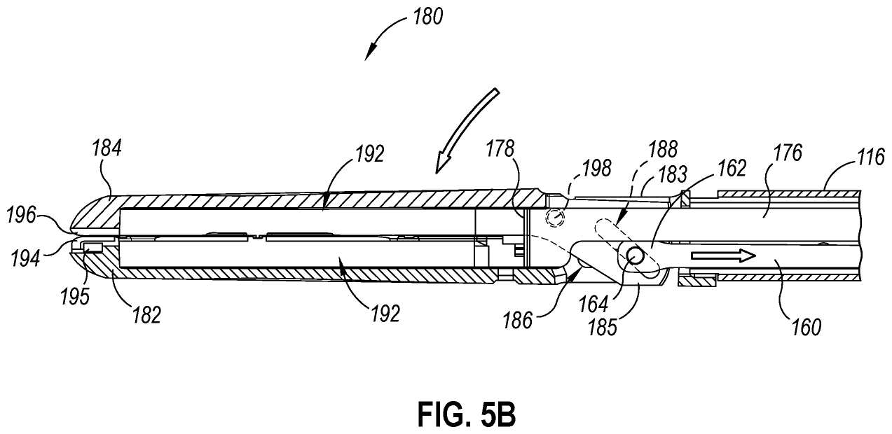

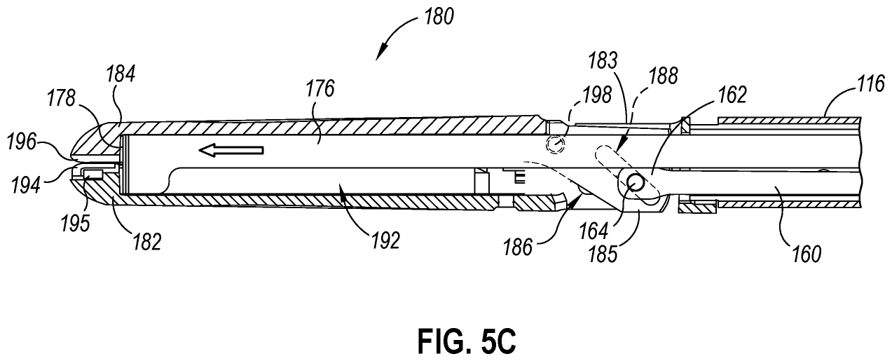

As best seen in FIGS. 2-3, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connecter (160), and pin (164). Slots (186, 188) each slidably receive pin (164), which is attached to a distal coupling portion (162) of jaw closure connector (160). Additionally, as best seen in FIGS. 5A-5C, end effector (180) includes a sensor assembly (195) located at a distal tip of lower jaw (182). Sensor assembly (195) may be in communication with control unit (102). Sensor assembly (195) may be configured to measure any suitable data as would be apparent to one skilled in the art in view of the teachings herein. For example, sensor assembly (195) may be configured to measure various characteristics of tissue grasped between jaws (182, 814). As another example, sensor assembly (195) may be configured to measure various characters of jaws (182, 184) during exemplary use, such as closure force or a closure angle of jaws (182, 184) relative to each other. As another example, sensor assembly (195) may include an articulation sensor or feedback mechanism which measures the degree end effector (180) is deflected from the longitudinal axis (LA) by articulation control (132) and articulation section (110). Additionally, sensor assembly (195) may communicate this data to control unit (102). Any suitable components may be used for force sensor (195) as would be apparent to one skilled in art in view of the teachings herein. For example, force sensor (195) may take the form of a strain gauge.

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted (as shown in FIGS. 5A-5B), and advanced (as shown in FIG. 5C), to cut tissue captured between jaws (182, 184). Lower jaw (182) and upper jaw (184) each comprise a respective electrode surface (194, 196). The power source may provide RF energy to electrode surfaces (194, 196) via electrical coupling (15) that extends through handle assembly (120), shaft assembly (140), articulation assembly (110), and electrically couples with one or both of electrode surfaces (194, 196). Electrical coupling (15) may selectively activate electrode surfaces (194, 196) in response to an operator pressing activation button (130). In some instances, control unit (102) may couple electrical coupling (15) with activation button (130), such that control unit (102) activates electrode surfaces (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc.

FIGS. 4A-5C show an exemplary use of instrument (100) for end effector (180) to grasp, cut, and seal/weld tissue. As described above, and as shown between FIGS. 4A-4B and 5A-5B, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

Handle assembly (120) further includes a yoke assembly (200) that is slidably coupled along proximal portion (144) of shaft assembly (140). Yoke assembly (200) is operatively coupled with jaw closure connector (160) such that translation of yoke assembly (200) relative to proximal portion (144) of shaft assembly (140) translates jaw closure connector (160) relative to shaft assembly (140).

As best seen in FIGS. 4A-4C, yoke assembly (200) is coupled to a body (150) of jaw closure trigger (126) via a link (154). Link (154) is pivotally coupled with yoke assembly (200) via pin (156); while link (154) is also pivotally coupled with body (150) of jaw closure trigger (126) via pin (152). Additionally, jaw closure trigger (126) is pivotally coupled with body (122) of handle assembly (120) via pin (170). Therefore, as shown between FIGS. 4A-4B, an operator may pull jaw closure trigger (126) toward pistol grip (124), thereby rotating jaw closure trigger (126) about pin (170). Rotation of jaw closure trigger (126) leads to rotation of link (154) about both pins (152, 156), which in turn drives yoke assembly (200) in the proximal direction along proximal portion (144) of shaft assembly (140).

As described above, jaw closure connector (160) extends within shaft assembly (140), articulation section (110), and central channel (190) of lower jaw (182). As also mentioned above, jaw closure connector (160) is attached to pin (164). Therefore, as seen between FIGS. 5A-5B, proximal translation of yoke assembly (200) leads to proximal translation of pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

As best seen in FIGS. 4A-4C, yoke assembly (200) is also coupled with a bias spring (155). Bias spring (155) is also coupled to a portion of body (122), such that bias spring (155) biases yoke assembly (200) to the position shown in FIG. 4A (associated with the open configuration of end effector (180) as shown in FIG. 5A). Therefore, if an operator releases jaw closure trigger (126), bias spring (155) will translate yoke assembly (200) to the position shown in FIG. 4A, thereby opening jaws (182, 184) of end effector (180).

As described above, and as shown between FIGS. 4B-4C and 5B-5C, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body (174) that is slidably coupled along proximal portion (144) of shaft assembly (140). Knife coupling body (174) is coupled with knife member (176) such that translation of knife coupling body (174) relative to proximal portion (144) of shaft assembly (140) translates knife member (176) relative to shaft assembly (140).

As best seen in FIGS. 4B-4C and 5B-5C, knife coupling body (174) is coupled a knife actuation assembly (168) such that as knife trigger (128) pivots toward body (122) and/or pistol grip (124), knife actuation assembly (168) drives knife coupling body (174) distally, thereby driving knife member (176) distally within knife pathway (192). Because knife coupling body (174) is coupled to knife member (176), knife member (176) translates distally within shaft assembly (140), articulation section (110), and within knife pathway (192) of end effector (180), as best shown between FIGS. 5B-5C. Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

Knife trigger (128) is biased to the positions seen in FIGS. 4A-4B (associated with the knife member (176) in the retracted position) by a bias arm (129). Bias arm (129) may include any suitable biasing mechanism as would be apparent to one having ordinary skill in the art in view of the teachings herein. For instance, bias arm (129) may include a torsion spring. Therefore, if an operator releases knife trigger (128), bias arm (129) returns knife trigger (128) to the position shown in FIGS. 4A-4B, thereby translating knife member (176) toward the retracted position.

With distal cutting edge (178) of knife member (176) actuated to the advance position (position shown in FIG. 5C), an operator may press activation button (130) to selectively activate electrode surfaces (194, 196) of jaws (182, 184) to weld/seal severed tissue that is captured between jaws (182, 184). It should be understood that the operator may also press activation button (130) to selectively activate electrode surfaces (194, 196) of jaws (182, 184) at any suitable time during exemplary use. Therefore, the operator may also press activation button (130) while knife member (176) is retracted as shown in FIGS. 3A-3B. Next, the operator may release jaw closure trigger (128) such that jaws (182, 184) pivot into the opened configuration, releasing tissue.

### II. Exemplary Electrosurgical Instrument with Fiber Optic Rotary Coupling

As mentioned above, end effector (180) includes a sensor assembly (195) that may be configured to measure various characteristics of tissue grasped between jaws (182, 184), or other suitable characteristics as would be apparent to one skilled in the art in view of the teachings herein. In some instances, it may be desirable to provide a sensor assembly (195) that utilizes fiber optic capabilities in order to obtain and transmit data accumulated at or near end effector (180). In such instances, one or more fiber optic cables may extend proximally from sensor assembly (195), through shaft assembly (140), and through handle assembly (120) in order to suitably couple with a fiber optic compatible device. Such a fiber optic compatible device (also known as a “lightbox”) may be configured to generate a source of light to be transmitted through fiber optic cables, as well as interpret data via light obtained from sensor assembly (195) and transmitted through fiber optic cables. Examples of such fiber optic compatible devices with one or more features that may be incorporated into end effector (180) are described U.S. Pat. App. No. [Atty. Ref. END9391USNP1], entitled “Electrosurgical System with Optical Sensor Electronics,” filed on even date herewith and U.S. Pat. App. No. [Atty. Ref END9392USNP1], entitled “Electrosurgical Instrument with Light Accumulator End Effector and Fiber Optics,” filed on even date herewith. The disclosure of each of these US patent documents is incorporated by reference herein.

However, utilizing fiber optic cables has limitations. For instance, if a fiber optic cable is bent with too small a bend radius, the light transmitted through that fiber optic cable (and therefore the data contained within that light) may become too inefficient or otherwise degraded in order to provide accurate and/or meaningful data. Therefore, if a fiber optic cable were utilized with sensor assembly (195), and such a cable had too small a bend radius along its length, the data obtained from sensor assembly (195) may not be suitably transmitted to a lightbox. Fiber optic cables generally come with a manufacturer's recommended predetermined minimum bend radius for a specific fiber optic cable, such that fiber optic cables may suitably transmit light as long as all the bends along the length of the cable are larger than the predetermined minimum bend radius.

As mentioned above, electrosurgical instrument (100) includes a knob (134) that is rotatably disposed on body (122) and configured to rotate end effector (180) (which includes sensor assembly (195)) and shaft assembly (140) about the longitudinal axis (LA) of shaft assembly (140) relative to handle assembly (120). As also mentioned above, in instances where fiber optics are incorporated into sensor assembly (195), a fiber optical cable may extend from sensor assembly (195), through shaft assembly (140), and through handle assembly (120) into a lightbox. In such instances, when end effector (180) is rotated about the longitudinal axis (LA) relative to handle assembly (120) in accordance with the description herein, a proximal portion of a fiber optic cable connected to sensor assembly (195) may rotate relative to a distal portion of fiber optic cable connected to the lightbox. Relative rotation between a fiber optic cable may render the fiber optic cable inoperable and/or damage to the fiber optic cable by (A) bending the cable with a bend radius smaller than the predetermined minimum bend radius, (B) inducing undesirable torsional forces in the cable, or (C) any other undesirable means as would be apparent to one skilled in the art in view of the teachings herein.

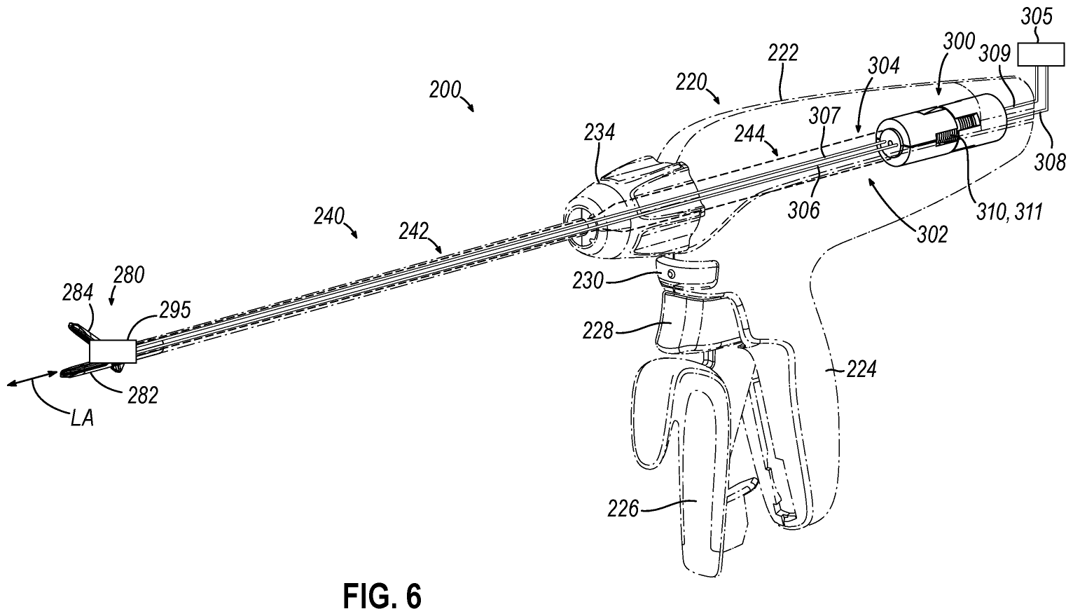

Therefore, when incorporating fiber optic cables in electrosurgical instrument (100), it may be desirable to provide a fiber optic rotational coupling assembly configured to allow rotation of a distal end of a fiber optic cable about the longitudinal axis (LA) relative to a proximal end of the fiber optic cable coupled to the lightbox without rendering the fiber optic cable inoperable or damaging the fiber optic cable. FIG. 6 shows an exemplary electrosurgical instrument (200) that is substantially similar to electrosurgical instrument (100) described above, with differences elaborated below. In particular, electrosurgical instrument (200) includes optical fiber cables (302, 304) and a fiber optic rotational coupling assembly (300). Fiber optic cables (302, 304) extend from a lightbox (305), through fiber optic rotational coupling assembly (300) housed within handle assembly (320), and couple with sensor assembly (295). As will be described in greater detail below, Fiber optic rotational coupling assembly (330) allows for a distal portion (306, 307) of fiber optic cables (302, 304) to rotate with shaft assembly (240) about longitudinal axis (LA) relative to both body assembly (220) and a proximal portion (308, 309) of fiber optic cables (302, 304) without (A) damaging fiber optic cables (302, 304), and (B) bending cables (302, 304) with a bend radius smaller than the predetermined minimum bend radius and thereby inhibiting the ability of cables (302, 304) to suitably transmit light in accordance with the description herein.

Electrosurgical instrument (200) includes a handle assembly (220), a body (222), a pistol grip (224), a jaw closure trigger (226), a knife trigger (228), an actuation button (230), a knob (234), a shaft assembly (240), a distal portion (242), a proximal portion (242), an end effector (280), a pair of jaws (282, 284), and a sensor assembly (295); which may be substantially similar to handle assembly (120), body (122), pistol grip (124), jaw closure trigger (126), knife trigger (128), actuation button (130), knob (134), shaft assembly (140), distal portion (142), proximal portion (142), end effector (180), jaws (182, 184), and sensor assembly (195) described above, with differences elaborated below. Electrosurgical instrument (200) may also include an articulation assembly (not shown) and an articulation control (not shown); which may be substantially similar to articulation assembly (110) and articulation control (132) described above, respectively.

Sensor assembly (295) is configured to work in conjunction with fiber optic cables (302, 304) and lightbox (305) in order to provide meaningful information related to end effector (280) and/or the environment around end effector (280). In some instances, a first (or first group of) fiber optic cable(s) (302, 304) may transmit light from lightbox (305) to sensor assembly (295) such that sensor assembly may distribute light generated by lightbox (305) to end effector (280); while a second (or second group of) fiber optic cable(s) (302, 304) may collect light distributed from sensor assembly (295) and transmit the collected light back to lightbox (305). Lightbox (305) may then process the light provided by the second fiber optic cable (302, 304) in order to provide meaningful data relevant to the components and/or area located adjacent to sensor assembly (295). In other words, one cable (302, 304) may transmit light to sensor assembly (295) such that sensor assembly (295) may distribute that light to the surrounding area adjacent to sensor assembly (295); while a second cable (302, 304) may collect light reflected by the surrounding area adjacent to sensor assembly (295).

Each fiber optic cable (302, 304) includes a distal portion (306, 307) associated with shaft assembly (240), a proximal portion (308, 309) extending from lightbox (305) into handle assembly (220), and a coiled section (310, 311) interposed between distal portion (306, 307) and proximal portion (308, 309). Distal portions (306, 307) extend from rotational coupling assembly (300) within shaft assembly (240) and terminates distally into sensor assembly (295); while proximal portions (308, 309) of cable (302, 304) are associated with handle assembly (320) and extend proximally to suitably couple with lightbox (305). Distal portions (306, 307) are configured to rotate with sensor assembly (295) and shaft assembly (240) relative to handle assembly (320) and proximal portions (308, 309) of cables (302, 204) about the longitudinal axis (LA) defined by shaft assembly (240) in response to rotation of knob (234) relative to body assembly (220). Distal portions (306, 307) may be laterally spaced away from the longitudinal axis (LA) defined by shaft assembly (240).

Coiled sections (310, 311) are housed within fiber optic rotational coupling assembly (300) and interposed between distal portion (306, 307) and proximal portion (308, 309). As will be described in greater detail below, rotational coupling assembly (300) is configured to accommodate rotation of distal portions (306, 307) relative to proximal portions (308, 309) of respective cables (302, 304) without damaging cables (302, 304) or inhibiting the ability of cables (302, 304) to suitably transmit light in accordance with the description herein. In particular, rotational coupling assembly (300) is configured to controllably and predictably radially expand and contract coiled sections (310, 311) to accommodate rotation of distal portion (306, 307) relative to proximal portion (308, 309) through a predetermined range of angular displacement about the longitudinal axis (LA).

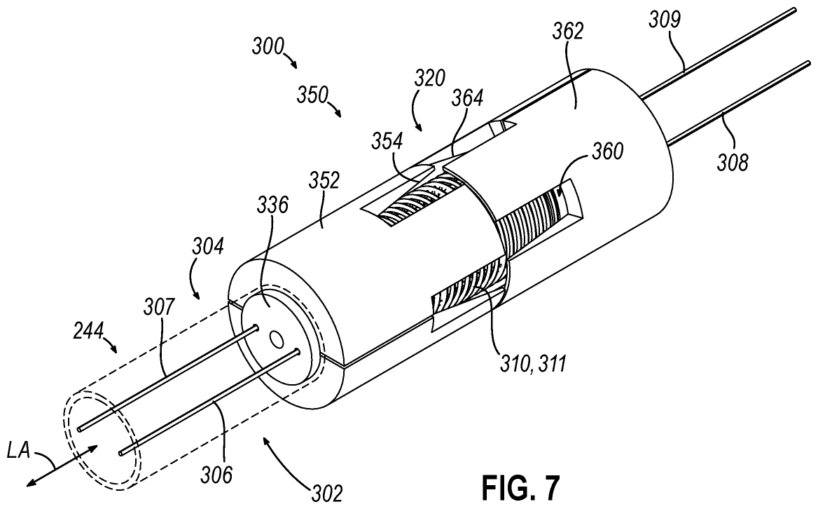

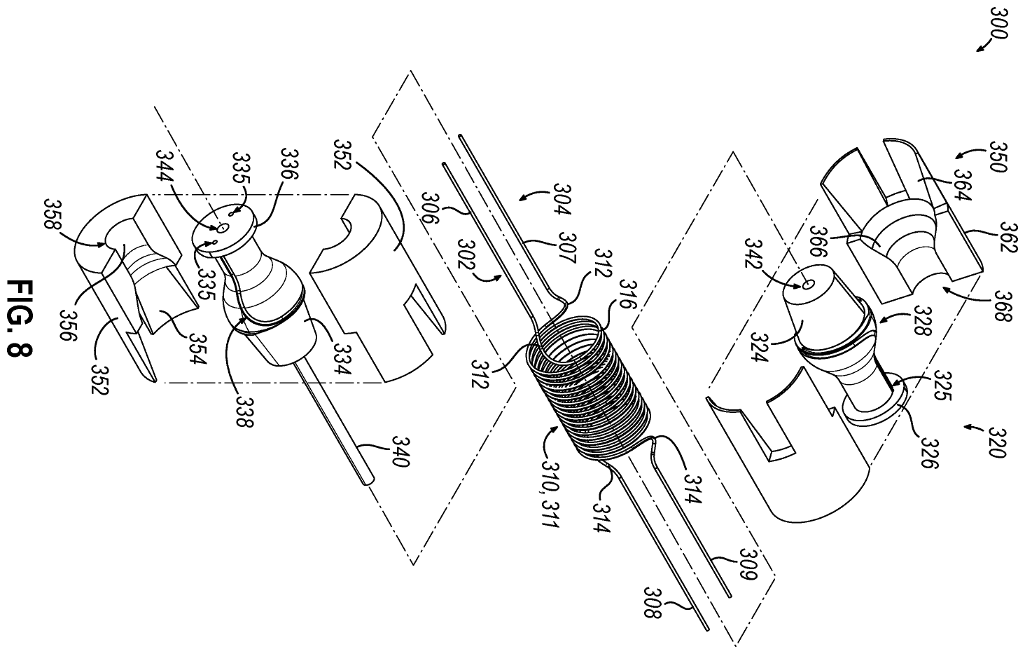

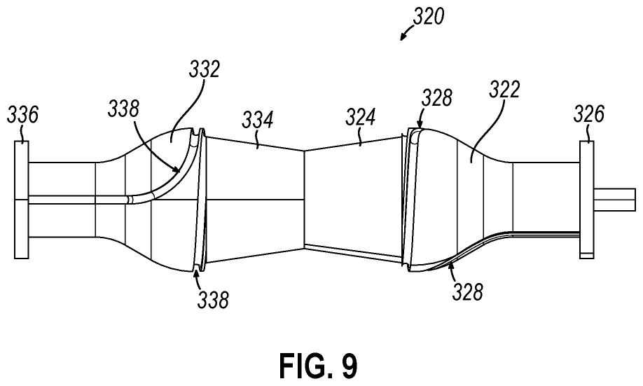

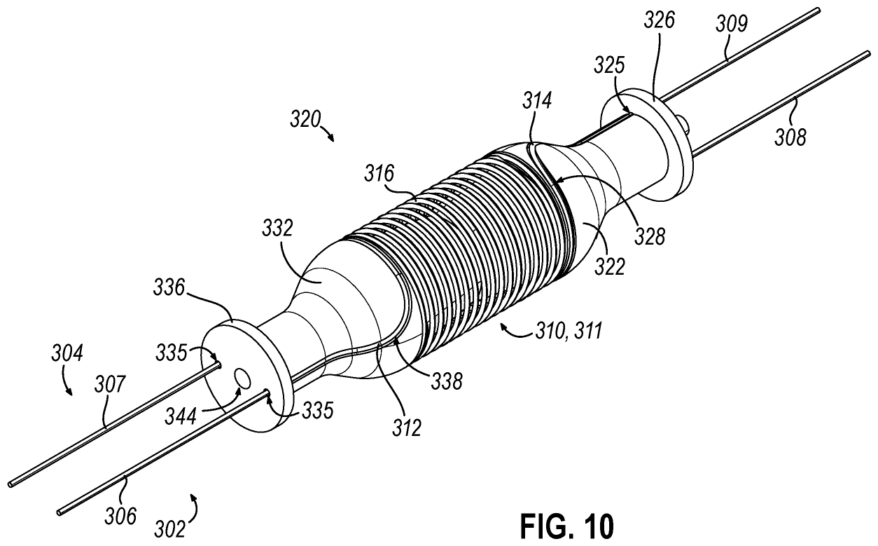

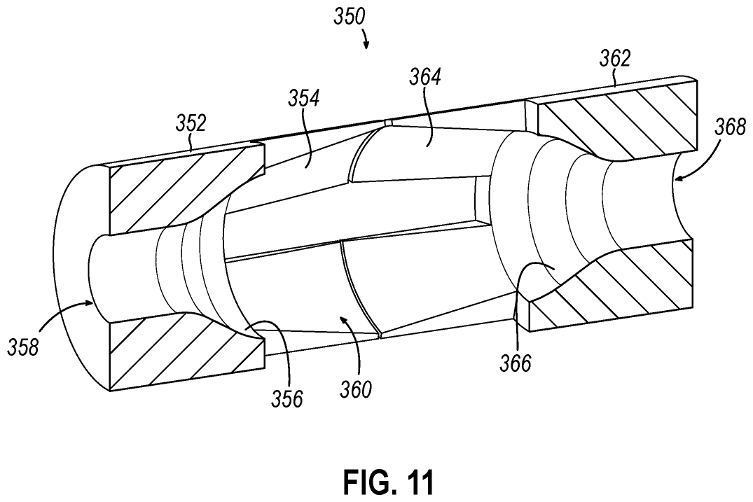

Turning to FIGS. 7-8, fiber optic rotational coupling assembly (300) includes an inner spindle assembly (320) (shown assembled in FIGS. 9-10) and a housing assembly (350) (shown assembled in FIG. 11). Inner spindle assembly (320) includes a handle-side body (322) and a shaft-side body (332). Handle-side body (322) and shaft-side body (332) are rotationally coupled to each via a pair of rotary coupling features (340, 342), which include a rod (340) and through hole (342) in the current example. Through hole (342) is dimensioned to receive rod (340) such that shaft side body (332) may rotate relative to handle-side body (322) about the longitudinal axis (LA) defined by shaft assembly (240). In some instances, rod (342) may be hollow such that other components may be fed through the hollow portion of rod (342) along the length of inner spindle assembly (320). While in the current example, rod (340) and through hole (342) are used to rotatably couple handle-side body (322) and shaft-side body (332), this is merely optional. Any other rotational coupling means may be utilized as would be apparent to one skilled in the art in view of the teachings herein.

Handle-side body (322) is attached to handle assembly (220) such that handle-side body (322) may be substantially rotationally fixed to body (222) of handle assembly (220) about the long axis of inner spindle assembly (320). Handle-side body (322) includes a tapered conical surface (324) and a proximal collar (326). A portion of handle-side body (322) extending between proximal collar (326) and conical surface (324) defines a pair of cable channels (328) dimensioned to receive a respective fiber optic cable (302, 304). A proximal portion of channel (328) extends into a respective through hole (325) defined by collar (326) such that proximal portion (308, 309) of cables (302, 304) may extend through collar (326) and into a respective channel (328).

As best shown in FIG. 10, cable channels (328) extend along handle-side body (322) such that the portion of fiber optic cable (302, 304) extending within channel (328) defines a proximal bend radius (314). Channel (328) accommodates a proximal bend radius (314) that is larger than the predetermined minimum bend radius of fiber optic cables (302, 304). Therefore, the portion of fiber optic cables (302, 304) extending within channel (328) may effectively communicate light in accordance with the description herein. Additionally, the portion of fiber optic cables (302, 304) housed within channels (328) may be substantially fixed within their respective channel (328). As will be described in greater detail below, interaction between channels (328) and portions of fiber optic cables (302, 304) housed within channels (328) may help radially contract and expand coiled sections in response to relative rotation of shaft-side body (332) and handle-side body (322).

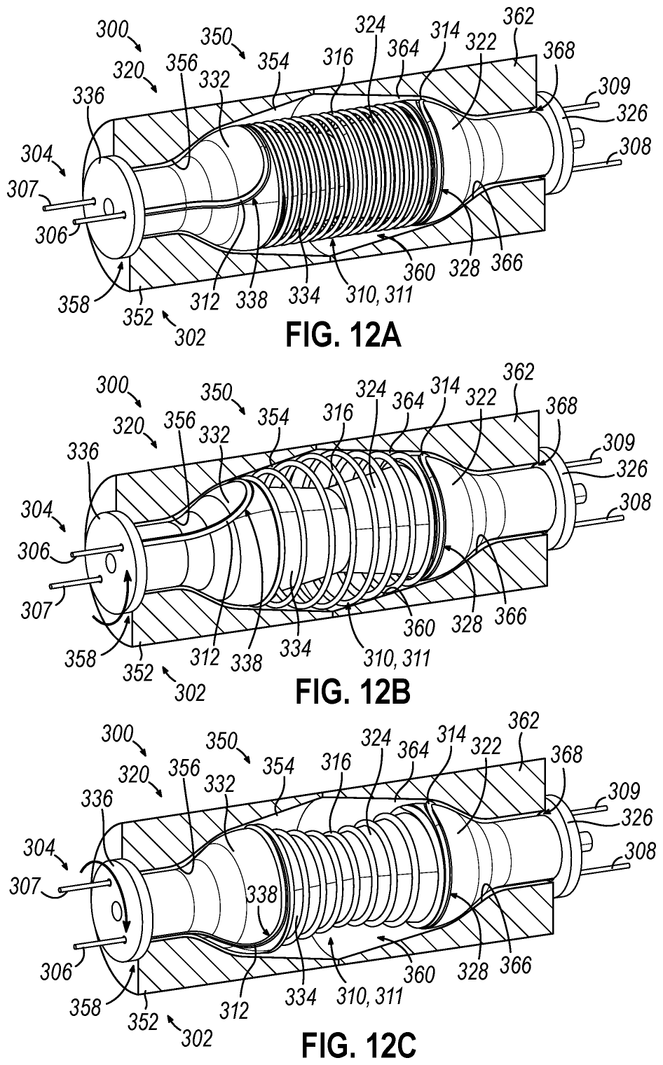

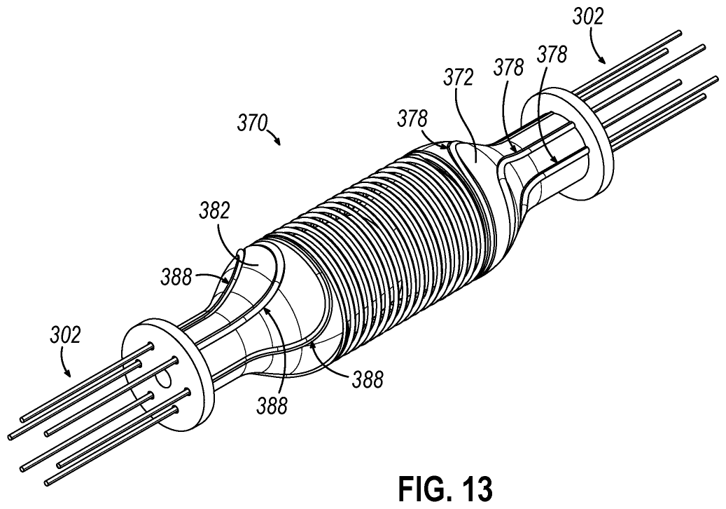

The radius of tapered conical surface (324) decreases as surface (324) extends distally from the rest of handle-side body (322) such that the smallest radius of tapered conical surface (324) is located near a distal end of handle-side body (322). As will be described in greater detail below, tapered conical surface (324) is dimensioned to abut against coiled section (310, 311) of fiber optic cables (302, 304) when distal portions (306, 307) of cables (302, 304) are rotated with shaft assembly (240) in a second angular direction at a second angular displacement limit (see FIG. 12C) about the longitudinal axis (LA). The smallest radius of tapered conical surface (324) is larger than the predetermined minimum bend radius of fiber optic cables (302, 304). Therefore, the portion of fiber optic cables (302, 304) abutting against tapered conical surface (324) may still effectively communicate light in accordance with the description herein when shaft assembly (240) is rotated in accordance with the description herein.

Shaft-side body (332) is attached to shaft assembly (240) such that shaft-side body (332) may rotate with shaft assembly (240) relative to body assembly (220) and handle-side body (322) in accordance with the teachings herein. shaft-side body (332) includes a tapered conical surface (334) and a distal collar (336). A portion of shaft-side body (332) extending between distal collar (336) and conical surface (334) defines a pair of cable channels (338) dimensioned to receive a respective fiber optic cable (302, 304). A distal portion of channel (338) extends into a respective through hole (335) defined by collar (336) such that distal portion (306, 307) of cables (302, 304) may extend within through collar (336).

As best shown in FIG. 10, cable channels (338) extend along shaft-side body (332) such that the portion of fiber optic cable (302, 304) extending within channel (338) defines a distal bend radius (312). Channel (338) accommodates a distal bend radius (312) that is larger than the predetermined minimum bend radius of fiber optic cables (302, 304). Therefore, the portion of fiber optic cables (302, 304) extending within channel (338) may effectively communicate light in accordance with the description herein. In some instances, the distal bend radius (312) is the same as the proximal bend radius (314).

Additionally, the portion of fiber optic cables (302, 304) housed within channels (338) may be substantially fixed within their respective channel (338) such that as shaft-side body (332) rotates relative to handle-side body (322), the portion of cables (302, 304) within channels (338) of shaft-side body (332) also rotate relative to the potion of cable (302, 304) within channels (328) of handle-side body (322). As will be described in greater detail below, this relative rotation between portions of cables (302, 304) fixed within channel (328, 338) helps radially contract and expand coiled sections in response to relative rotation of shaft-side body (332) and handle-side body (322).

The radius of tapered conical surface (334) decreases as surface (334) extends proximally from the rest of handle-side body (322) such that the smallest radius of tapered conical surface (324) is located near a proximal end of shaft-side body (332). Similar to tapered conical surface (324), tapered conical surface (334) is dimensioned to abut against coiled section (310, 311) of fiber optic cables (302, 304) when distal portions (306, 307) of cables (302, 304) are rotated with shaft assembly (240) in the second angular direction at the second angular displacement limit (see FIG. 12C) about the longitudinal axis (LA). Additionally, the smallest radius of tapered conical surface (334) is larger than the predetermined minimum bend radius of fiber optic cables (302, 304). Therefore, the portion of fiber optic cables (302, 304) abutting against tapered conical surface (334) may still effectively communicate light in accordance with the description herein when shaft assembly (240) is rotated in accordance with the description herein.

As best shown in FIGS. 8 and 11, housing assembly (350) of fiber optic rotational coupling assembly (300) includes a pair of distal housing bodies (352) and a pair of proximal housing bodies (362). Distal housing bodies (352) are configured to couple with each other in order to house shaft-side body (332), while proximal housing bodies (362) are configured to couple with each other in order to house handle-side body (322). Distal housing bodies (352) and proximal housing bodies (362) each includes a complementary interior surface (356, 366) dimensioned to house portions of inner spindles assembly (320) defining channels (328, 338).

Once suitably coupled, distal housing bodies (352) define distal portion (358) while proximal housing bodies (362) define a proximal opening (368). Together, all housing bodies (352, 362) define an interior chamber (360) dimensioned to house inner spindle assembly (320) (besides collars (326, 336) and coiled sections (310, 311) of fiber optics cables (302, 304). As best shown in FIGS. 12A-12B, openings (358, 368) are dimensioned smaller than collars (336, 326) such that when assembled, inner spindles assembly (320) is substantially longitudinally fixed relative to housing assembly (350). In some instances, distal housing bodies (352) are configured to rotate with shaft-side body (332) of inner spindle assembly (320). In other instances, distal housing bodies (352) may be rotationally fixed relative to handle assembly (220). Proximal housing bodies (362) may be substantially fixed relative to handle-side body (322).

Tapered interior surfaces (354, 364) are angled such that surfaces expand (354, 364) laterally outward as surfaces (354, 364) extend toward each other. As will be described in greater detail below, tapered interior surfaces (354, 364) are dimensioned to abut against coiled section (310, 311) of fiber optic cables (302, 304) when distal portions (306, 307) of cables (302, 304) are rotated with shaft assembly (240) in a first angular direction at a second angular displacement limit about the longitudinal axis (LA).

FIGS. 12A-12C show an exemplary use of fiber optic rotational coupling assembly (300) in order to rotate shaft assembly (240) relative to handle assembly (220) such that distal portions (306, 307) of cables (302, 304) may rotate relative to the longitudinal axis (LA) of shaft assembly (240) and proximal portion (308, 309) of cables (302, 304) without (A) damaging fiber optic cables (302, 304), and (B) bending cables (302, 304) with a bend radius smaller than the predetermined minimum bend radius and thereby inhibiting the ability of cables (302, 304) to suitably transmit light in accordance with the description herein. FIG. 12A shows shaft-side body (332) of inner spindle assembly (320) in a neutral rotational position such that coiled sections (310, 311) define a first coiled bend radius (316). While defining the first coiled bend radius (316), coiled sections (310, (311) wrap around tapered conical surfaces (324, 334) without contacting neither conical surface (324, 334) or tapered interior surfaces (354, 364) of housing assembly (350).

Next, during exemplary use as shown in FIG. 12B, an operator may desire to rotate end effector (280) relative to body assembly (220) into a desired angular position about the longitudinal axis (LA) of shaft assembly (240). Therefore, the operator may rotate shaft assembly (240) and end effector (280) in accordance with the description herein in the first angular direction, and if desired, all the way to a first angular displacement limit about the longitudinal axis (LA) as shown in FIG. 12B.

In response to rotation of shaft assembly (240) and end effector (280), shaft-side body (332) of inner spindle assembly (320) may rotate relative to handle-side body (322) such that portions of fiber optic cables (302, 304) housed within respective channels (328, 338) rotate relative to each other in the first angular direction. As a result of this rotation, coiled sections (310, 311) may be driven to radially expand such that coiled section (310, 311) now have a larger coiled bend radius (316) as compared to when in inner spindle assembly (320) is in the neutral position as shown in FIG. 12A.

Shaft assembly (240) may be rotated from the neutral position in the first angular direction all the way to the first angular displacement limit shown in FIG. 12B such that coiled sections (310, 311) abut against tapered interior surfaces (354, 364). It should be understood that the controlled expansion of coiled section (310, 311) may allow for rotation of shaft assembly (240) in the first angular direction all the way to the first angular displacement limit without damaging cables (302, 304) or inhibiting the ability of cables (302, 304) to transmit light and/or data during exemplary use.

Next, during exemplary use as shown in FIG. 12C, an operator may desire to rotate end effector (280) relative to body assembly (220) into another desired angular position about the longitudinal axis (LA) of shaft assembly (240). Therefore, the operator may rotate shaft assembly (240) and end effector (280) in accordance with the description herein in the second angular direction, and if desired, all the way to a second angular displacement limit about the longitudinal axis (LA) as shown in FIG. 12C.

In response to rotation of shaft assembly (240) and end effector (280), shaft-side body (332) of inner spindle assembly (320) may rotate relative to handle-side body (322) such that portions of fiber optic cables (302, 304) housed within respective channels (328, 338) rotate relative to each other in the second angular direction. As a result of this rotation, coiled sections (310, 311) may be driven to radially contract such that coiled section (310, 311) now have a smaller coiled bend radius (316) as compared to when in inner spindle assembly (320) is in the neutral position as shown in FIG. 12A.

Shaft assembly (240) may be rotated from the neutral position in the second angular direction all the way to the second angular displacement limit shown in FIG. 12B such that coiled sections (310, 311) abut against tapered conical surfaces (324, 334). As mentioned above, the smallest radius of tapered conical surfaces (324, 334) are larger than the predetermined minimum bend radius of fiber optic cables (302, 304). Therefore, the portion of coiled sections (310, 311) abutting against tapered conical surfaces (324, 334) may still effectively communicate light in accordance with the description herein, even when shaft assembly (240) is rotated in the second angular direction into the second angular displacement limit. It should be understood that the controlled contraction of coiled section (310, 311) may allow for rotation of shaft assembly (240) in the second angular direction all the way to the second angular displacement limit without damaging cables (302, 304) or inhibiting the ability of cables (302, 304) to transmit light and/or data during exemplary use.

In some instances, rotational coupling assembly may include a rotational lockout assembly configured to inhibit rotation of shaft assembly (240) past the first and second angular displacement limits when rotated in the first and second angular directions, respectively. Therefore, coiled sections (310, 311) of cables (302, 304) may be prevented from radially expanding and contracting past the positions shown in FIGS. 12B and 12C, respectively. Any suitable rotational lockout features may be incorporated as would be apparent to one skilled in the art in view of the teachings herein.

In the current example, instrument (200) is shown with two fiber optic cables (302, 304) such that inner spindle assembly (320) is configured to accommodate for two fiber optic cables (302, 304). However, it should be understood that instrument (200) and inner spindle assembly (320) may be configured to use any suitable number of fiber optic cables (302, 304) as would be apparent to one skilled in the art in view of the teachings herein.

FIG. 13 shows an alternative inner spindle assembly (370) that may readily incorporated into fiber optic rotational coupling assembly (300) in replacement of inner spindle assembly (320) described above. Inner spindle assembly (370) is substantially similar to inner spindle assembly (300) described above, with differences elaborated below. Therefore, inner spindle assembly (370) includes a handle-side body (372) and a shaft-side body (382) which are substantially similar to handle-side body (322) and shaft-side body (332) described above, with differences elaborated below. In particular, handle-side body (372) and a shaft-side body (382) each define six cable channels (378, 388), rather than two. Therefore, bodies (372, 382) are both configured to receive six fiber optic cables (302) rather than two fiber optic cables (302, 304). Inner spindle assembly (370) is configured to accommodate rotation of shaft assembly (240) relative to body assembly (220) while coupling six fiber optic cables (302) between lightbox (305) and sensor assembly (295) such that inner spindle assembly (370) may controllably and predictably radially expand and contract coiled portions of six fiber optic cables (302).

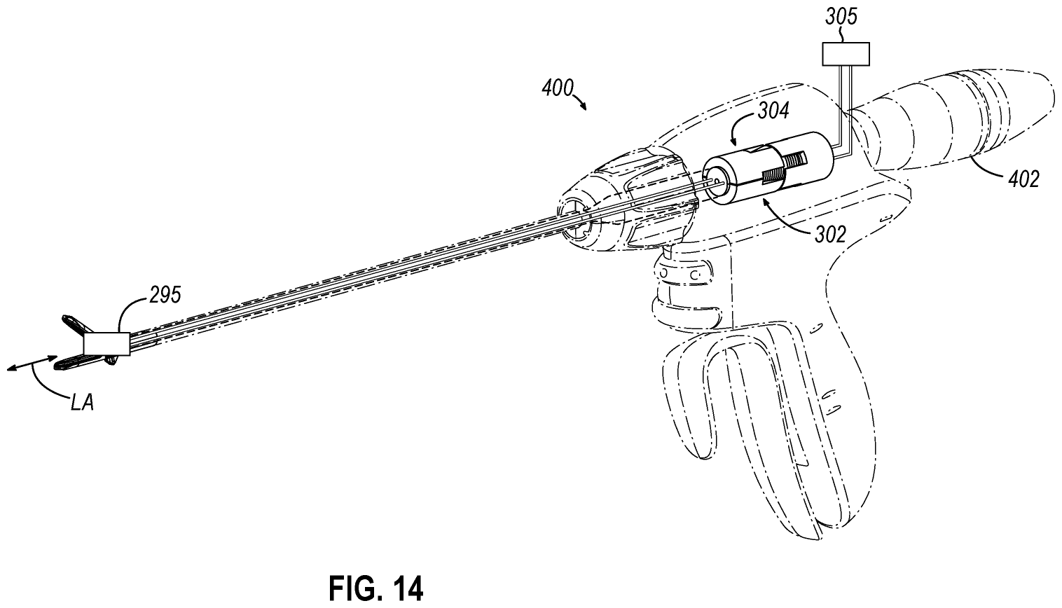

In the current example, fiber optic rotational coupling (300) is used in conjunction with instrument (200) having an end effector (280) configured to deliver RF energy to tissue grasped between jaws (282, 284). However, it should be understood that fiber optic rotational coupling (300) may be used in conjunction with any other suitable instrument configured to deliver any other suitably type of energy to tissue as would be apparent to one skilled in the art in view of the teachings herein. FIG. 14 shows fiber optic rotational coupling (300) incorporated into an instrument (400) with an ultrasonic transducer (402). As mentioned above, in some instances, rod (342) may be hollow such that other components may be fed through the hollow portion of rod (342) along the length of inner spindle assembly (320). In instances where an ultrasonic transducer is incorporated, an ultrasonic waveguide may be fed through the hollow portion of rod (342). Therefore, ultrasonic vibrations may be communicated from ultrasonic transducer (402), through rotational coupling (300) and an ultrasonic blade via an ultrasonic waveguide extending through coupling (300).

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A surgical instrument, comprising: (a) a body; (b) a shaft assembly extending distally from the body, wherein the shaft assembly extends along a longitudinal axis, wherein the shaft assembly is configured to rotate relative to the body about the longitudinal axis; (c) an end effector extending distally from the shaft assembly, wherein the end effector is configured to rotate with the shaft assembly relative to the body about the longitudinal axis, wherein the end effector comprises a sensor assembly; (d) a first fiber optic cable extending proximally from the sensor assembly along the shaft assembly and into the body, wherein the first fiber optic cable comprises: (i) a distal portion configured to rotate with the end effector, (ii) a proximal portion associated with the body such that the distal portion is configured to rotate relative to the proximal portion, and (ii) a coiled portion interposed between the distal portion and the proximal portion; and (e) a rotary coupling assembly comprising: (i) a handle-side body associated with the proximal portion of the first fiber optic cable, and (ii) a shaft-side body rotatably coupled with the handle-side body, wherein the shaft-side body is associated with the distal portion of the first fiber optic cable, wherein the rotary coupling assembly is configured to radially expand and contract the coiled portion in response to relative rotation between the handle-side body and the shaft-side body.

### EXAMPLE 2

The surgical instrument of any one or more of the preceding Examples, wherein the rotary coupling assembly further comprise a coupling feature configured to rotatably couple the handle-side body with the shaft-side body.

### EXAMPLE 3

The surgical instrument of any one or more of the preceding Examples, wherein the coupling feature comprises a rod associated with the shaft-side body.

### EXAMPLE 4

The surgical instrument of any one or more of the preceding Examples, wherein the coupling feature comprises a through hole defined by the handle-side body.

### EXAMPLE 5

The surgical instrument of any one or more of the preceding Examples, wherein the handle-side body defines a proximal cable channel, wherein a section of the proximal portion of the first fiber optic cable is housed within the proximal cable channel.

### EXAMPLE 6

The surgical instrument of any one or more of the preceding Examples, wherein the shaft-side body defines a distal cable channel, wherein a section of the distal portion of the first fiber optic cable is housed within the distal cable channel.

### EXAMPLE 7

The surgical instrument of any one or more of the preceding Examples, wherein the handle-side body comprises a first tapered conical surface.

### EXAMPLE 8

The surgical instrument of any one or more of the preceding Examples, wherein the shaft-side body comprises a second tapered conical surface.

### EXAMPLE 9

The surgical instrument of any one or more of the preceding Examples, wherein the first tapered conical surface and the second tapered conical surface abut against each other.

### EXAMPLE 10

The surgical instrument of any one or more of the preceding Examples, wherein the coiled portion of the first fiber optic cable is configured to abut against the first tapered conical surface and the second tapered conical surface in a first angular position such that the coiled portion of the first fiber optic cable is inhibited from reaching a predetermined minimum bend radius of the first fiber optic cable.

### EXAMPLE 11

The surgical instrument of any one or more of the preceding Examples, wherein the rotary coupling assembly further comprising a housing, wherein at least a portion of the handle-side body and the shaft-side body are contained within the housing.

### EXAMPLE 12

The surgical instrument of any one or more of the preceding Examples, wherein the housing comprises a tapered interior surface, wherein the coiled section is configured to abut against the tapered interior surface in a second angular position.

### EXAMPLE 13

The surgical instrument of any one or more of the preceding Examples, further comprising a second fiber optic cable attached to the rotary coupling assembly.

### EXAMPLE 14

The surgical instrument of any one or more of the preceding Examples, wherein the end effector is configured to deliver RF energy to tissue.

### EXAMPLE 15

The surgical instrument of any one or more of the preceding Examples, wherein the body comprises a handle assembly.

### EXAMPLE 16

A surgical instrument, comprising: (a) a body; (b) a shaft assembly extending distally from the body, wherein the shaft assembly extends along a longitudinal axis, wherein the shaft assembly is configured to rotate relative to the body about the longitudinal axis; (c) an end effector extending distally from the shaft assembly, wherein the end effector is configured to rotate with the shaft assembly relative to the body about the longitudinal axis, wherein the end effector comprises a sensor assembly; (d) a fiber optic cable assembly extending proximally from the sensor assembly along the shaft assembly and into the body, wherein the fiber optic cable assembly comprises: (i) a distal portion attached to the sensor assembly, (ii) a proximal portion attached to the body, and (ii) a coiled portion interposed between the distal portion and the proximal portion; and (e) a rotary coupling assembly comprising: (i) a handle-side assembly associated with the proximal portion of the first fiber optic cable, and (ii) a shaft-side assembly rotatably coupled with the handle-side assembly such that the rotary coupling assembly is configured to radially expand and contract the coiled portion in response to relative rotation between the handle-side body and the shaft-side body.

### EXAMPLE 17

The surgical instrument of any one or more of the preceding Examples, wherein the fiber optic cable assembly comprises six fiber optic cables.

### EXAMPLE 18

The surgical instrument of any one or more of the preceding Examples, wherein the handle-side assembly comprises a proximal collar defining a plurality of through holes.

### EXAMPLE 19

The surgical instrument of any one or more of the preceding Examples, wherein the shaft-side assembly comprises a distal collar attached to the shaft assembly.

### EXAMPLE 20

A surgical instrument, comprising: (a) a fiber optic cable comprising: (i) a distal portion, (ii) a proximal portion, and (ii) a coiled portion interposed between the distal portion and the proximal portion; and (b) a rotary coupling assembly comprising: (i) a first body associated with the proximal portion of the fiber optic cable, and (ii) a second body rotatably coupled with the first body, wherein the second body is associated with the distal portion of the fiber optic cable, wherein the rotary coupling assembly is configured to radially expand and contract the coiled portion in response to relative rotation between the first body and the second body.

### IV. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Additionally, any one or more of the teachings herein may be combined with any one or more of the teachings of U.S. Pat. App. No. [Atty. Ref. END9391USNP1], entitled “Electrosurgical System with Optical Sensor Electronics,” filed on even date herewith; and U.S. Pat. App. No. [Atty. Ref END9392USNP1], entitled “Electrosurgical Instrument with Light Accumulator End Effector and Fiber Optics,” filed on even date herewith. The disclosure of each of these US patent documents is incorporated by reference herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. A surgical instrument, comprising:
(a) a body;
(b) a shaft assembly extending distally from the body, wherein the shaft assembly extends along a longitudinal axis, wherein the shaft assembly is configured to rotate relative to the body about the longitudinal axis;
(c) an end effector extending distally from the shaft assembly, wherein the end effector is configured to rotate with the shaft assembly relative to the body about the longitudinal axis, wherein the end effector comprises a sensor assembly;
(d) a first fiber optic cable extending proximally from the sensor assembly along the shaft assembly and into the body, wherein the first fiber optic cable comprises:
(i) a distal portion configured to rotate with the end effector,
(ii) a proximal portion associated with the body such that the distal portion is configured to rotate relative to the proximal portion, and
(ii) a coiled portion interposed between the distal portion and the proximal portion; and
(e) a rotary coupling assembly comprising:
(i) a handle-side body associated with the proximal portion of the first fiber optic cable, and
(ii) a shaft-side body rotatably coupled with the handle-side body, wherein the shaft-side body is associated with the distal portion of the first fiber optic cable, wherein the rotary coupling assembly is configured to radially expand and contract the coiled portion in response to relative rotation between the handle-side body and the shaft-side body.

2. The surgical instrument of claim 1, wherein the rotary coupling assembly further comprises a coupling feature configured to rotatably couple the handle-side body with the shaft-side body.

3. The surgical instrument of claim 2, wherein the coupling feature comprises a rod associated with the shaft-side body.

4. The surgical instrument of claim 3, wherein the coupling feature comprises a through hole defined by the handle-side body.

5. The surgical instrument of claim 1, wherein the handle-side body defines a proximal cable channel, wherein a section of the proximal portion of the first fiber optic cable is housed within the proximal cable channel.

6. The surgical instrument of claim 5, wherein the shaft-side body defines a distal cable channel, wherein a section of the distal portion of the first fiber optic cable is housed within the distal cable channel.

7. The surgical instrument of claim 1, wherein the handle-side body comprises a first tapered conical surface.

8. The surgical instrument of claim 7, wherein the shaft-side body comprises a second tapered conical surface.

9. The surgical instrument of claim 8, wherein the first tapered conical surface and the second tapered conical surface abut against each other.

10. The surgical instrument of claim 9, wherein the coiled portion of the first fiber optic cable is configured to abut against the first tapered conical surface and the second tapered conical surface in a first angular position such that the coiled portion of the first fiber optic cable is inhibited from reaching a predetermined minimum bend radius of the first fiber optic cable.

11. The surgical instrument of claim 10, wherein the rotary coupling assembly further comprising a housing, wherein at least a portion of the handle-side body and the shaft-side body are contained within the housing.

12. The surgical instrument of claim 11, wherein the housing comprises a tapered interior surface, wherein the coiled section is configured to abut against the tapered interior surface in a second angular position.

13. The surgical instrument of claim 1, further comprising a second fiber optic cable attached to the rotary coupling assembly.

14. The surgical instrument of claim 1, wherein the end effector is configured to deliver RF energy to tissue.

15. The surgical instrument of claim 1, wherein the body comprises a handle assembly.

16. A surgical instrument, comprising:
(a) a body;
(b) a shaft assembly extending distally from the body, wherein the shaft assembly extends along a longitudinal axis, wherein the shaft assembly is configured to rotate relative to the body about the longitudinal axis;
(c) an end effector extending distally from the shaft assembly, wherein the end effector is configured to rotate with the shaft assembly relative to the body about the longitudinal axis, wherein the end effector comprises a sensor assembly;
(d) a fiber optic cable assembly extending proximally from the sensor assembly along the shaft assembly and into the body, wherein the fiber optic cable assembly comprises:
(i) a distal portion attached to the sensor assembly,
(ii) a proximal portion attached to the body, and
(ii) a coiled portion interposed between the distal portion and the proximal portion; and
(e) a rotary coupling assembly comprising:
(i) a handle-side assembly associated with the proximal portion of the first fiber optic cable, and
(ii) a shaft-side assembly rotatably coupled with the handle-side assembly such that the rotary coupling assembly is configured to radially expand and contract the coiled portion in response to relative rotation between the handle-side body and the shaft-side body.

17. The surgical instrument of claim 16, wherein the fiber optic cable assembly comprises six fiber optic cables.

18. The surgical instrument of claim 16, wherein the handle-side assembly comprises a proximal collar defining a plurality of through holes.

19. The surgical instrument of claim 18, wherein the shaft-side assembly comprises a distal collar attached to the shaft assembly.

20. A surgical instrument, comprising:
(a) a fiber optic cable comprising:
(i) a distal portion,
(ii) a proximal portion, and
(ii) a coiled portion interposed between the distal portion and the proximal portion; and
(b) a rotary coupling assembly comprising:
(i) a first body associated with the proximal portion of the fiber optic cable, and
(ii) a second body rotatably coupled with the first body, wherein the second body is associated with the distal portion of the fiber optic cable, wherein the rotary coupling assembly is configured to radially expand and contract the coiled portion in response to relative rotation between the first body and the second body.
